(19) United States
(12) Patent Application Publication
(10) Pub. No.: US 2013/0079590 A1
(43) Pub. Date: Mar. 28, 2013

(54) MULTIPURPOSE SURGICAL SPONGEPADS, DEVICES, SYSTEMS, AND METHODS

(71) Applicant: BRADLEY P. BENGTSON, Grand Rapids, MI (US)

(72) Inventor: BRADLEY P. BENGTSON, Grand Rapids, MI (US)

(21) Appl. No.: 13/425,971

(22) Filed: Mar. 21, 2012

Related U.S. Application Data
(60) Provisional application No. 61/454,840, filed on Mar. 21, 2011.

(51) Int. Cl. A61F 13/15; A61M 37/00; A61B 1/32; A61F 13/53; A61F 13/84

(52) U.S. Cl. CPC A61F 13/15268; A61F 13/53; A61F 13/8405; A61B 1/32; A61M 37/00
USPC 600/37; 604/385.15; 604/372; 604/360; 604/93.01; 435/284.1

(57) ABSTRACT

Novel surgical sponge-pad devices that are highly absorbent, durable, and reusable are presented. The technical features of the surgical sponge-pad devices make possible unique surgical sponge-pad systems and methods that take advantage of the use, conditioning, and reuse of a single surgical sponge device again and again during a surgical procedure. The invention provides surgical-sponge-pad devices, systems, and methods that make possible a significant reduction in the number of surgical sponges needed for a given surgical procedure. In addition this multifunctional surgical sponge-pad will have significant and brand new applications in surgery for the protection and hydration of tissues, transmission of antibiotics and antimicrobials and other solutions, barriers in surgery and even use in transfer of transplanted organs as well as many other future medical and surgical uses as yet undefined and undiscovered.

### RELATED APPLICATIONS

This application claims the benefit of provisional application Ser. No. 61/454,840 filed 21 Mar. 2011.

### FIELD OF THE INVENTION

The invention generally relates to devices, systems, and methods for performing surgical procedures, and more particularly, temporary, non implantable surgical sponge-pad devices, systems, and methods for the absorption of blood and body fluids, temporary packings, hydration of tissues, tissue protection, organ transportation, dressings and the like in an operative site used during medical and surgical procedures.

### BACKGROUND OF THE INVENTION

A surgical sponge 10 (see FIG. 1A) is a specialized sponge or pad used in surgery. Standard surgical sponges 10 of various shapes and sizes have long been used in a diverse number of surgical procedures for the absorption of blood and body fluids, packings, and the like. Also commonly known as a “lap sponge” or “Raytec sponge”, a surgical sponge 10 is used to absorb liquids from a surgical site. Surgical sponges 10 may be used in combination with suction to keep a surgical site clear by a surgeon working to control bleeding or absorb fluid (as FIG. 1A shows), and in a number of other applications. As shown in FIGS. 1B and 2, it is a common surgical technique to use surgical forceps 12 to manipulate a surgical sponge 10 in an operative field or sponges may be used manually to dab and wick away blood and fluid from the operative field. They are also commonly used to wall-off bowel or lung or other organs away from the surgical field to get better exposure to the specific area of focus during the surgical procedure.

Like other things used in surgery, surgical sponges 10 are designed to be sterile. They come in their own sterile packaging and can be safely autoclaved or terminally sterilized in multiple ways. Standard Lap sponges and Raytec also commonly carry lint and other particles and though they may be sterile, the material may come off and be retained inside the body or wound. In addition Raytec and lap sponges can be very abrasive and traumatize the skin and tissues.

Materials used in conventional surgical sponges 10 range from cotton, rayon and polyesters, from gauzes to non-wovens. Conventional surgical sponges are available in various sizes: e.g., 12″×12″ and upwards to 24″×24″; or 4″×18″ and upwards to 8″×36″ when they are open completely.

During a typical surgical procedure, a relatively large number of surgical sponges 10, e.g., exceeding thirty to forty, may be required. Conventional surgical sponges 10 are treated as single use items and disposed of after blood soaked or used because they become nonfunctional and become impregnated with blood and a patient's body fluids. As shown in FIG. 2, surgical sponges 10 are used once during surgery and then discarded in a biohazard container 14, which can also hold other disposable surgical instruments and equipment. The sheer number of surgical sponges 10 that are used a single time and then discarded during a given surgical procedure contributes to environmental damage due to increased medical waste (as FIG. 2 demonstrates), as well as increases the overall cost of health care.

The sheer number of surgical sponges 10 that are conventionally used during a single surgical procedure also compounds the problem of accountability. Several techniques have been developed, e.g., counting all sponges 10 and instruments, sometimes with the assistance of a barcode scanner, or radio opaque markers, or radio-frequency identification devices (RFID), to reconcile the sponge count before wound closure, with the sponge count reconciliation at the end of a large surgical case often taking up to five minutes and the patient cannot be closed until the sponge needle and instrument count is correct. Inadvertent errors may occur, with a sponge 10 left within a surgical site when the wound is closed, termed a retained sponge. Sponges left behind may cause infection and serious complications. When the error is discovered, the surgeon must re-expose the surgical site and retrieve the sponge 10, causing undue further stress and trauma to the patient. In addition there are millions of dollars of litigation annually for a surgeon leaving a retained sponge inside a body cavity. Some institutions are now performing X-ray examination on all major cases where a chest or abdominal cavity is opened to further check for a retained sponge which also adds to operative time and exposes the patient to radiation.

### TECHNICAL FEATURES OF THE INVENTION

The invention provides a new surgical-pad with a nearly unlimited number of new and improved uses that overcome many of the problems and limitations current sponges have in surgery. One specific application for our present invention would be for a surgical sponge-pad substitute. Our device, however, is up to 1500% more absorbent, much more durable, lint and particle free, washable and reusable within the same surgical case reusable. The surgical pad may even be used to clean instruments or placed as a dressing on the patient following the end of the procedure. The technical features of the surgical-pad, surgical sponge devices make possible unique surgical pad, sponge systems and methods that take advantage of the use, conditioning, and reuse of a single surgical sponge device again and again during the same surgical procedure. The invention provides surgical sponge-pad devices, systems, and methods that make possible a significant reduction in the number of sponge-pads needed for a given surgical procedure compared to prior art sponges, e.g. 2-3 sponge-pads of the present invention versus 30-40 prior art sponges per case, and also markedly shortening the surgical time along with a marked reduction in the time required for a sponge count or unnecessary surgical delays.

The surgical sponge-pad devices comprise a sterilizable, liquid-absorbent, and durable material that can be economically sized and configured for use and reuse during a given surgical procedure. The material is dust and lint free (that is, it does not flake or shed fibers or the like). This new material is soft, non-abrasive and does not traumatize skin or deep tissue that is often very friable from a disease process such as infection or cancer. The material possesses an enhanced liquid absorbing capacity up to 1500%, which can be a very desirable characteristic, e.g., by a capability of holding at least ten times its weight in liquid. It can be used in the operating field, where the absorbent character of the material quickly absorbs blood, body fluids, as well as water and other aqueous liquids in an operative site. It may also be utilized to sequester off bowel to limit fecal spillage and contamination an infection in open bowel cases. The material is sized and configured to be quickly reconditioned outside the operating field, where the material can be quickly rinsed and cleaned by immersion in an aqueous liquid (e.g., saline) on the back table, and residual liquid easily released by manual wringing and rinsing in saline, and at that time be immediately available for return and reuse in the operating field. The material is sufficiently durable so that it can rinsed, cleaned, and wrung to release residual liquid repeatedly and returned for repeated reuse in the operating field, over and over again during the course of a single surgical procedure. At the end of the surgical procedure, the material can be disposed of as medical waste, with much less bioburden to our world.

One material that is particularly suited for use in the manner described is synthetic chamois. Such a material exhibits the above-noted desired characteristics; notably, an enhanced liquid absorbing and release/conditioning capacities, as well as the durability to withstand repeated reuse. A surgical sponge device comprising synthetic chamois can be repeatedly used to absorb blood, body fluids, as well as water and other aqueous liquids in an operative site, then quickly rinsed, wrung to release residual liquid, and returned for reuse in the operative field, again and again during the course of a given surgical procedure. Synthetic chamois can be made from, for example, polyvinyl alcohol, polyurethane, polyvinyl acetate, polypropylene, polyester, and polytetraflouroethylene (PTFE), polyglycolic acid and other like-based materials. Other suitable materials, such as polyester, cellulose materials, micro denier or other cleaning textiles, foam like materials or fabrics may also be used. The surgical sponge devices may also include coatings, e.g. anti-microbial and anti-fungal coatings, or soaked in such materials like antiseptics, antimicrobials and even antibiotic solutions.

The preferred materials for the surgical sponge-pad devices are highly absorbent compared to prior art sponges and pads. These new surgical sponge-pad devices have absorbency ranges of more than 100 percent increase of the traditional surgical sponges, to a 1000 percent increase absorbency compared to traditional sponges, and over 1500% increase in absorbency over some traditional sponges on the market and utilized in surgery every day. The surgical sponge-pad devices also have an increased absorption rate per area, with absorption rates of at least 0.70 g/in² and up to and above 5.00 g/in². This clinically equates to placing the pad on a table and placing a large amount of water on the table top with the water immediately being soaked into the pad-sponge.

A comparison of various surgical sponge devices according to the present invention and other commercially available non-surgical sponges is demonstrated in FIGS. 28 and 29. The individual sponges were cut to size and placed within fluid until the sponges reached full saturation. The sponges were then removed from the fluid and weighed.

The surgical sponge-pad devices just described make possible surgical sponge systems and methods that lead to a significant reduction in the number of surgical sponges needed for a given surgical procedure. The reduction in the number of sponge-pad device compared to prior art surgical sponges, in turn, makes possible significantly less environmental damage due to the reduction in medical waste, as well as contributing to markedly lower health care and disposable costs and bio burden. The systems and methods also lessen the problem of reconciling the surgical sponge count before wound closure, because there are fewer surgical sponge-pad devices to be accounted for again decreasing dramatically the operating times for sponge counts that may range over $100/minute.

The nature and scope of the invention may be best understood by reference to the following description, taken in conjunction with the accompanying drawings. Additional applications may include background material for vascular and microvascular anastomosis that may also be soaked in plain Lidocaine to help keep blood vessels from constricting helping to keep the vascular or microvascular anastomosis open. In addition, problems occur in surgery when open wounds maintain tissue hydration, as well as protecting the wound edges from retractors from general surgery procedures to cardiothoracic and chest procedures where the large chest wall retractors may be very traumatic to the wound edges and inhibit would healing. Also, in spine and Neurosurgery, the wicking action of the foam can absorb cerebrospinal fluid and keep the field dry so repairs and surgery may be performed more efficiently. Some additional applications will certainly arise and may include an improved transportation system for organ transplantation, plastic surgery applications of absorbing fluid from liposuction sites and as dressings following surgical procedures. A wide variety of liquids such as cardioplegic solution, antibiotics, antimicrobials, antiseptics, vasoconstrictors, vasodilators, wounds growth factors, clotting agents to name a few can be incorporated into the surgical pads. In addition the surgical pads will be valuable in the emergency room and for trauma patients to decrease bleeding and keep tissue well hydrated until surgical repair and treatment may be performed.

### BRIEF DESCRIPTION OF THE DRAWINGS

FIG. 1A is a perspective view of a conventional surgical sponge and its packaging.

FIG. 1B is a perspective view of a conventional surgical sponge carried at the end of a conventional surgical forceps.

FIG. 2 is a view showing the use of many conventional surgical sponges and their disposal as medical waste during the course of a typical surgical procedure and significant bioburden generated.

FIG. 3 is a surgical sponge-pad system for a given surgical procedure in accordance with the present invention, including just a few surgical sponge devices that embody features of the invention, demonstrating the significant reduction in the number of surgical sponges necessary for any one surgical procedure.

FIGS. 4 to 13 illustrate the use of the surgical-pad, sponge system shown in FIG. 3 during a surgical procedure, including the use of one surgical sponge device in the operative field to absorb blood, body fluids, and other aqueous liquids in an operating field, while another surgical sponge device is, outside the operating field, rinsed and wrung to remove residual liquid to be used next in the operating field, demonstrating in FIG. 13 the significant reduction in medical waste at the end of the surgical procedure.

FIG. 14 is an alternate surgical sponge system embodying features of the present invention, wherein the sponge-pads may further be dyed a particular color.

FIGS. 15 and 16 demonstrate the dying process of using the sponge-pad system of FIG. 14.

FIG. 17 demonstrates the use of a surgical sponge-pad according to the present invention, wherein the sponge-pad is being incorporated into a procedure involving a breast surgery.

FIG. 18 demonstrates the use of a sponge-pad according to the present invention internally during a surgery, wherein the sponges are capable of absorbing bodily fluids during the surgery.

FIG. 19 demonstrates the use of the sponge-pad according to the present invention as body tissue protector from the clamps and retractors that can cause significant damage during the course of surgery when using a surgical instrument, such as a sternotomy or rib retractor.

FIG. 20 demonstrates a further use of a sponge-pad as shown in FIG. 18, with the sponge-pad being used to retract an internal organ. Current lap-sponges are too thin to hold back bowel or internal organs, where the present surgical sponge-pads have significant thickness and resistance to be a markedly improved barrier.

FIGS. 21A and 21B demonstrate the use of the sponge according to the present invention during a microvascular anastomosis or vascular surgical procedure.

FIG. 22 demonstrates the sponge according to the present invention with an alternate shape. In fact the surgical pad devices may be cut and shaped to unlimited sizes and contours to solve any specific surgical application.

FIG. 23 shows the sponge-pad of FIG. 22 used during an abdominal procedure.

FIG. 24 shows the sponge-pad of FIG. 22 used during a breast procedure.

FIG. 25 shows the sponge-pad of the present invention being used to transport an organ for a surgical transplant procedure.

FIG. 26 shows the sponge-pad of the present invention being used for sterilizing medical equipment or tables.

FIG. 27 demonstrates further utility of the sponge-pad of the present invention for cleaning surgical instrumentation.

FIG. 28 provides a comparison of the absorption percentage and other characteristics of various surgical sponge devices according to the present invention and other commercially available non-surgical sponges.

FIG. 29 shows an absorption percentage comparison of various surgical sponge devices according to the present invention and other commercially available non-surgical sponges.

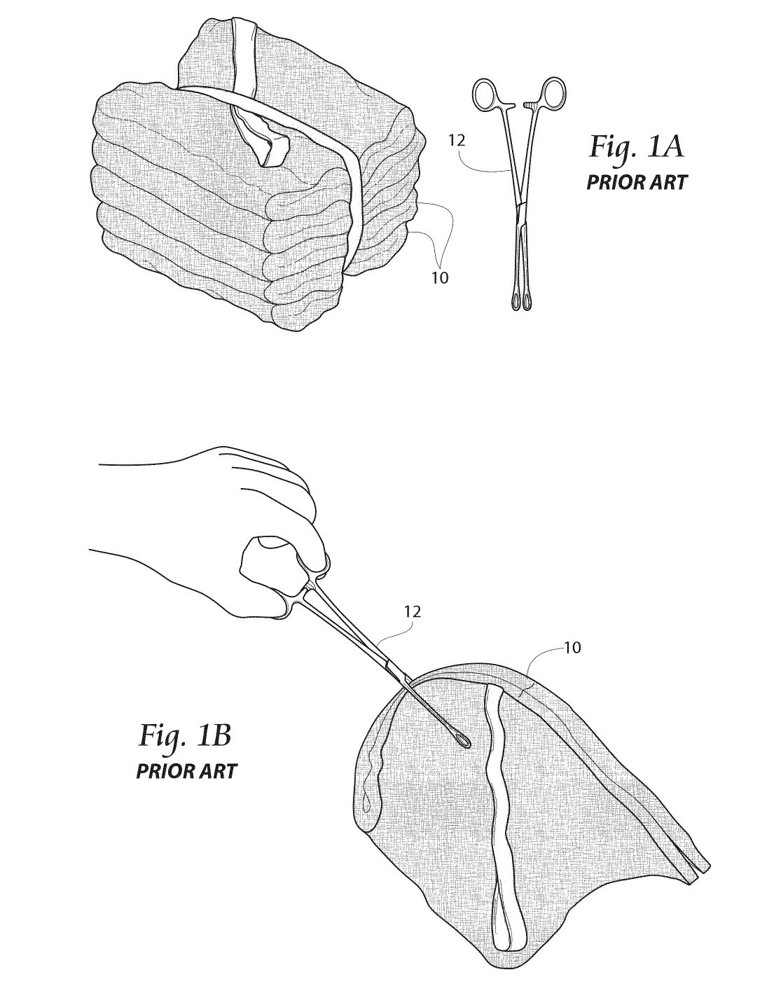

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

FIG. 3 shows a streamlined surgical sponge-pad system 16 for use during a single surgical procedure. The system 16 includes a prescribed, limited number of surgical sponge-pad devices 18, an aqueous conditioning liquid 20 carried in a container 22, and a conditioning tray 24. The streamlined surgical sponge system 16 also desirably includes instructions 26 for using the system 16.

Each surgical sponge-pad device 18 is sized and configured for use as a surgical sponge-pad depending on the specific use and application. The surgical sponge-pad devices 18 can be made available in various sizes, e.g., upwards to 24″×24″, 4″×18″, or 8″×36″, for manipulation in conventional ways, e.g., using surgical forceps 12. An example of a particular sized sponge device 18 would have the dimensions of 4″×10″×½, as further depicted in FIG. 5. Each surgical sponge device 18 can incorporate a radio opaque marker or RFID to aid in the reconciliation of the sponge count before wound closure or to be seen on X-ray if there is a question of one being retained inside a body cavity. The surgical sponge-pad device 18 may also incorporate a barium tag, which will assist locating the surgical sponge-pad device 18 when using during a procedure requiring an x-ray.

The surgical sponge-pad device 18 comprises surgical sponge material that is characterized according to the invention by a unique blend of physical characteristics that make it well suited for use and reuse as a surgical sponge in an operating room environment as well as a multifunctional device that is a single use and not designed to be retained inside the body, but disposed of following surgery or medical use. The material possesses a capability to quickly absorb blood, body fluids, as well as saline, water and other aqueous liquids found in or utilized in an operative site. The material also possesses the capability of being quickly rinsed clean after use by immersion in an aqueous solution such as sterile saline and residual liquid and or body fluids or blood and released by manual wringing. The material is sufficiently durable so that it can repeatedly rinsed, cleaned and wrung to release residual liquid and returned for reuse in an operating field.

Given these criteria, various materials can be identified by a person of ordinary skill in the art. A representative material having these characteristics is medical or surgical, intraoperative type of synthetic chamois. Synthetic chamois material is dust and lint free; that is, it does not flake or shed fibers or the like. Medical Synthetic chamois material can be sterilized, and it possesses a superior liquid absorbing capacity, e.g., a capability of holding up to fifteen times or greater (as noted above), its weight in liquid, and being up to 1500% more absorbent than current sponges used in surgery. Medical Synthetic chamois material is well suited for use in an operating environment, due to its capability to quickly absorb blood, body fluids, as well as water and other aqueous liquids in an operative site. Further, synthetic chamois material can be quickly reconditioned by immersion in a simple aqueous conditioning liquid (e.g., normal saline), where it can be quickly rinsed clean and then residual liquid easily released by manual wringing. After reconditioning, the surgical sponge-pad device 18 comprising synthetic chamois is immediately available to be returned for reuse in the operating field. The synthetic chamois material is sufficiently durable so that it can repeatedly rinsed cleaned and wrung to release residual liquid and returned for reuse in the operating field, over and over again during the course of a given surgical procedure. At the end of the surgical procedure, the synthetic chamois material can be economically disposed of as medical waste.

As will be evident from the various uses discussed below, the surgical sponge-pad device 18 not only can be reused during a surgical procedure, the same surgical sponge-pad device 18 can be used for differing procedures concurrently. The surgical sponge-pad device 18 can be sterilized during a surgical procedure and further be used for cleaning procedure, such as sterilizing the surgical equipment or operating table, which minimizes the number of different classifications of sponges or other devices that need to be supplied for an operating room or hospital. The surgical sponge-pad device 18 can be sterilized for intraoperative surgical uses, which greatly reduces waste and time, as the same surgical sponge-pad device 18 can be in multiple fashions.

Synthetic chamois can be made from, for example, polyvinyl alcohol, polyurethane, polyvinyl acetate and other like-based materials. Other suitable materials, such as polyester, micro denier or other cleaning textiles or fabrics may also be used.

Due to these beneficial characteristics, the surgical sponge-pad system 16 need include only a prescribed, limited number of surgical sponge-pad devices 18 for a given surgical procedure would be required. The limited number of surgical sponge-pad devices 18 selected for a given surgical procedure will vary according to the application and nature of the procedure and the number of personnel present in the operating room, anticipated amount of fluid production or bleeding. As will be described in greater detail later, as few as two surgical sponge-pad devices 18 may be required in an orderly repetition during a given surgical procedure. This stands in stark contrast to the numerous conventional surgical sponges that are typically deemed required by conventional medical wisdom.

In support of the limited number of surgical sponge-pad devices 18 provided, the surgical sponge-pad system 16 provides the aqueous conditioning liquid 20 (e.g., normal saline) for the surgical sponge-pad devices 18, which is shown packaged in a container 22 prior to use in the operating room (see FIG. 3). The surgical sponge-pad system 16 also provides the tray 24 to receive the conditioning liquid 20, and into which the surgical sponge-pad devices 18 are immersed for conditioning/reconditioning, as will be described in greater detail later. The conditioning liquid can include antibiotics, antimicrobials, antiseptics, haemostatic agents, chemotherapeutics, growth factors or other compounds.

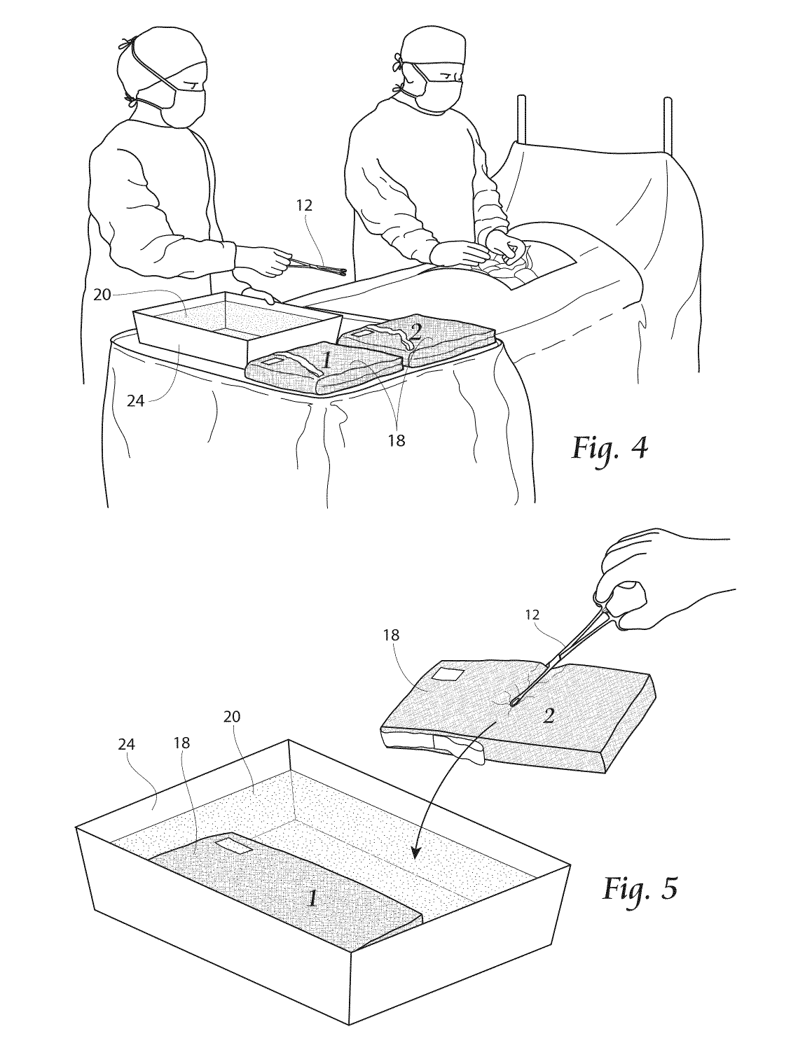

The unique functional features of each surgical sponge-pad device 18, as described, make possible a unique method of using only a limited number of surgical sponges during a given surgical procedure. FIGS. 4 to 13 show a representative method that makes use of these features. The streamlined surgical sponge-pad system 16 also desirably includes the instructions 26 for using the system according to the surgical sponge method as will be described.

The method includes (see FIG. 4) (i) providing the surgical sponge-pad system 16 comprising the limited number of surgical sponge-pad devices 18, the conditioning liquid 20, and the conditioning tray 24. These items can be provided in a sterilized kit, in the manner shown in FIG. 3. These items can be removed from the kit and set up on a prep table outside the operating field by an assistant (see FIG. 4). The assistant pours the conditioning liquid 20 into the tray 24. The assistant can also set out and arrange the limited number of surgical sponge devices 18 (numbered 1 and 2 in the drawings) on the table for use.

The method further includes (see FIG. 5) (ii) the assistant placing the surgical sponge devices 18 (numbered 1 and 2) in the conditioning liquid 20 to initially condition them for use. As FIG. 5 shows, the assistant can handle the surgical sponge devices 18 in a conventional way, using, e.g., surgical forceps 12.

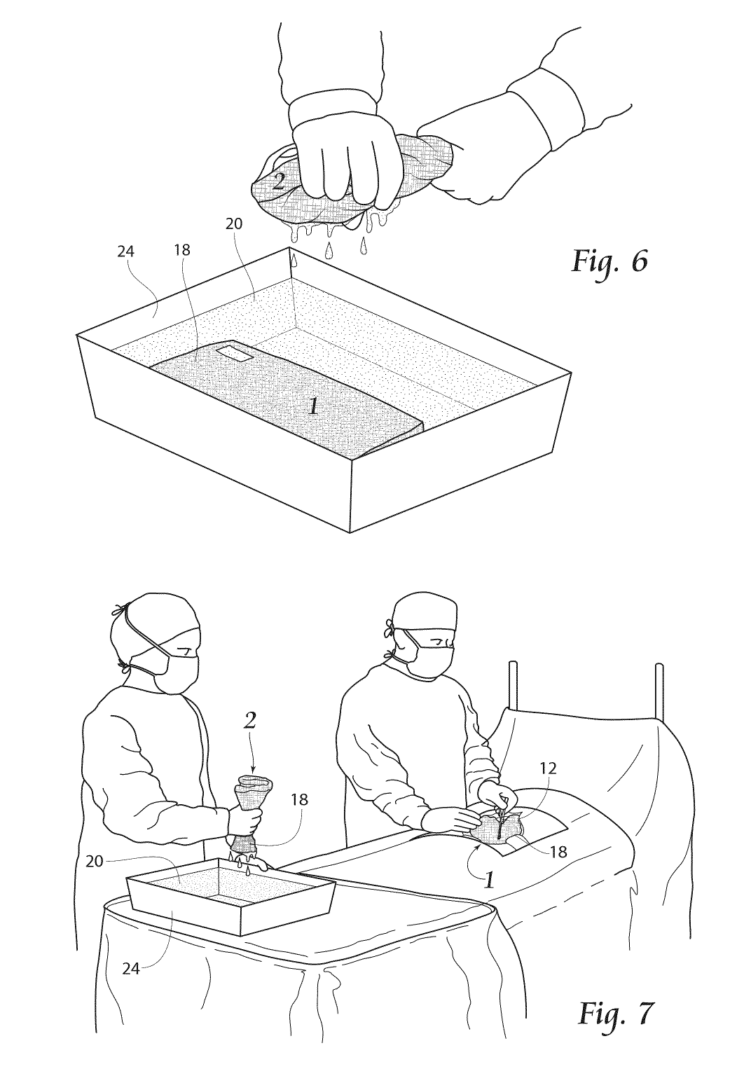

The method further includes (see FIG. 6) (iii) the assistant removing one of the surgical sponge-pad devices 18 (numbered 1) from the conditioning liquid 20 and releasing residual liquid by manual wringing. The method further includes (see FIG. 7) (iv) at the surgeon's request, the assistant handing the conditioned surgical sponge device 18 (numbered 1) to the surgeon (e.g., on surgical forceps) for use in the operating field. As FIG. 7 shows, the surgeon manipulates the surgical sponge-pad device 18 (numbered 1) in the operating field to absorb blood, body fluids, as well as water and other aqueous liquids in the operative site. Meanwhile, the method includes (as FIG. 7 also shows), (v) the assistant preparing the other surgical sponge-pad device 18 (numbered 2) for use by removing it from the conditioning liquid 20 and removing residual liquid by manual wringing.

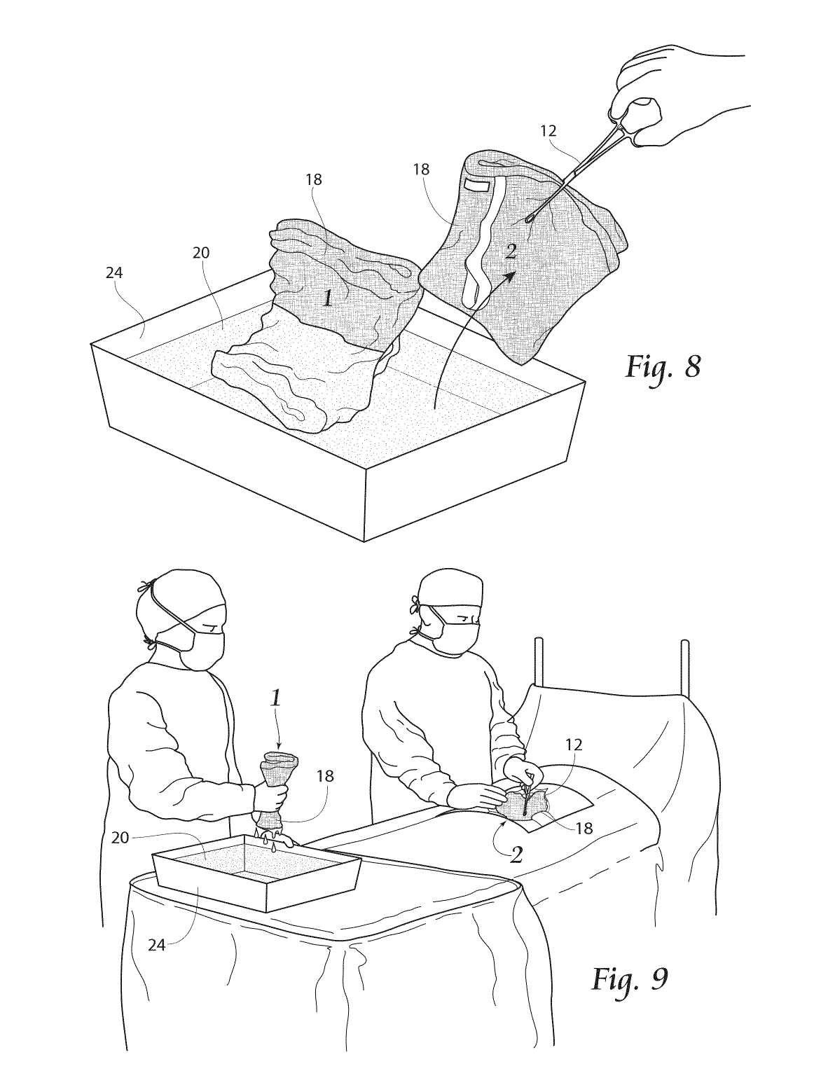

The method further includes (see FIG. 8) (vi) the surgeon handing the now-used surgical sponge-pad device 18 (numbered 1) to the assistant, who returns it to the conditioning liquid 20 to clean and rinse it. The method further includes (as FIG. 8 shows), (vii) at the surgeon's request, the assistant handing the previously wrung-out surgical sponge-pad device 18 (numbered 2) to the surgeon (e.g., on a surgical forceps 12) for use in the operating field.

As FIG. 9 shows, the method includes (viii) the surgeon manipulating the surgical sponge-pad device 18 (numbered 2) while (ix) the assistant prepares the other surgical sponge-pad device 18 (numbered 1) for reuse by removing it from the conditioning liquid 20 and manual wringing it to remove residual liquid. As will be appreciated further, the sponge-pad device 18 of the present invention can be used for multiple and varied uses.

As FIG. 10 shows, the method further includes (x) the surgeon handing the now-used surgical sponge-pad device 18 (numbered 2) to the assistant, who returns it to the conditioning liquid 20 to clean and rinse it. The method further includes (as FIG. 10 shows), (xi) at the surgeon's request, the assistant handing the previously wrung-out surgical sponge-pad device 18 (numbered 1) to the surgeon (e.g., on a surgical forceps) for reuse in the operating field.

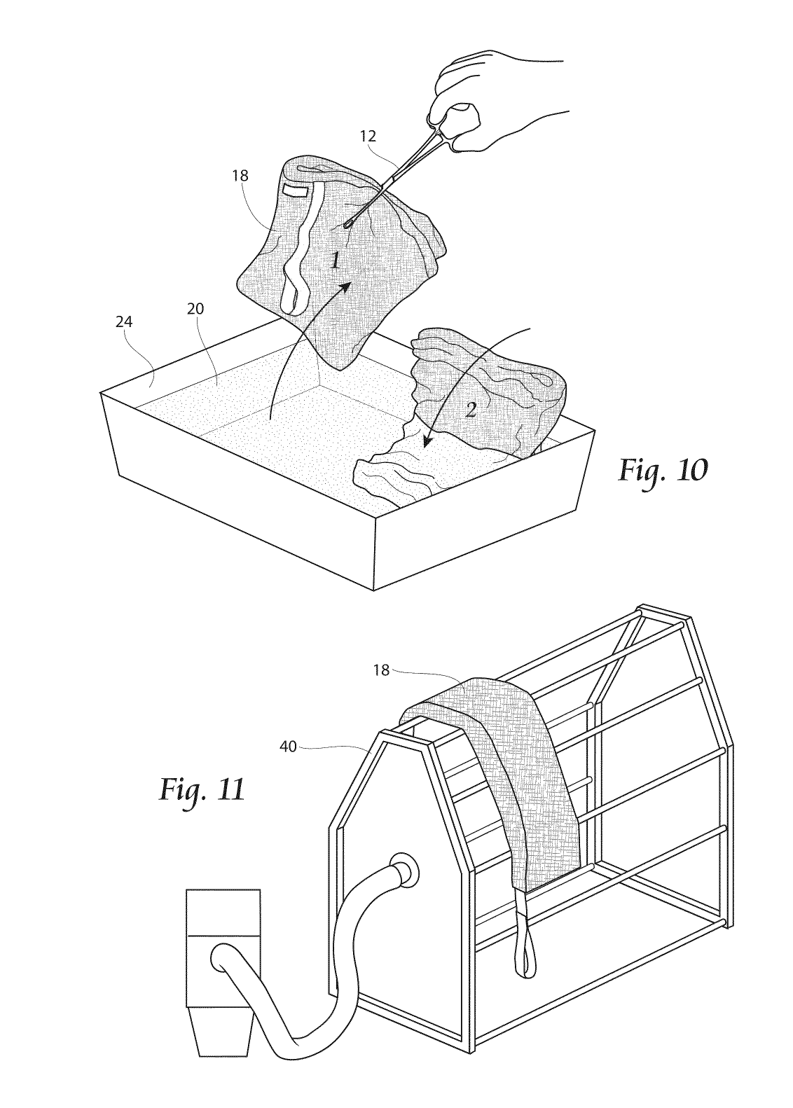

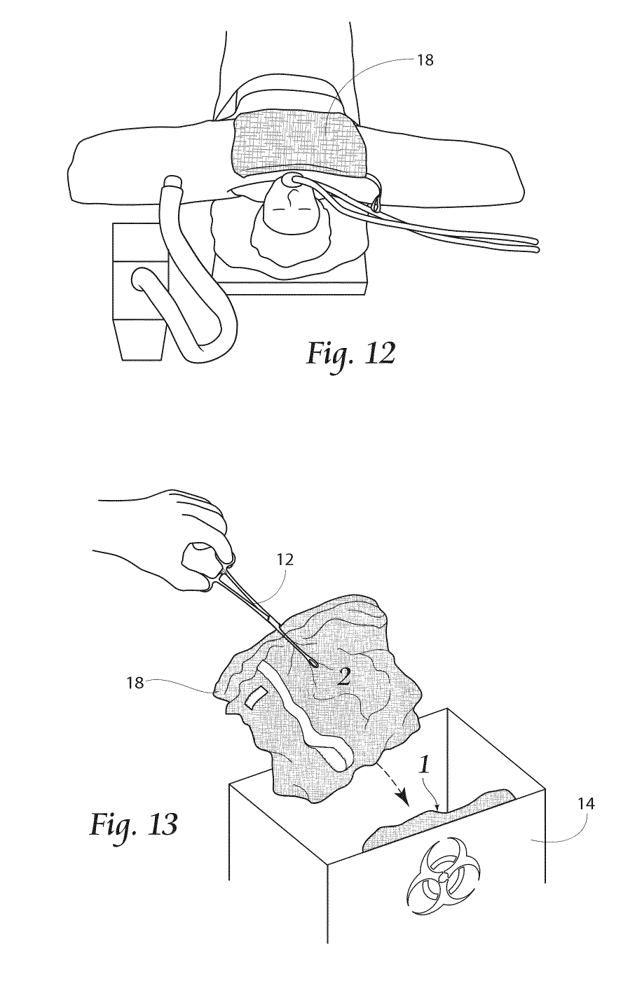

If necessary, the surgical sponge-pad devices 18 can be dried (xii) (see FIGS. 11 and 12). In FIG. 11, the sponge device 18 is dried in a conventional manner, wherein the sponge-pad device 18 is placed on a drying rack 40. The drying rack 40 is connected to a BAIR Hugger® (see FIG. 12), which will provide hot air to dry the sponge-pad device 18. Alternatively, in FIG. 12, an optional method of drying the sponge-pad device 18 is utilized, wherein the sponge-pad device 18 or devices 18 are placed on a drying rack with air from the BAIR Hugger® used to warm patients in surgery diverted to the drying rack to dry the sponges between usages. The thermal energy that would be typically released from the heating device 42 would be recycled to dry and warm the sponge-pad devices 18.

The above-described sequence is repeated as necessary through the remainder of the surgical procedure. While the surgeon reuses a surgical sponge-pad device 18 in the operating field, the assistant reconditions a previously used surgical sponge device 18 for reuse. The surgeon and assistant repeatedly exchange a currently used surgical sponge-pad device 18 for a reconditioned previously used surgical sponge-pad device 18, enabling each surgical sponge-pad device 18 to be reconditioned and reused again and again during the course of the procedure.

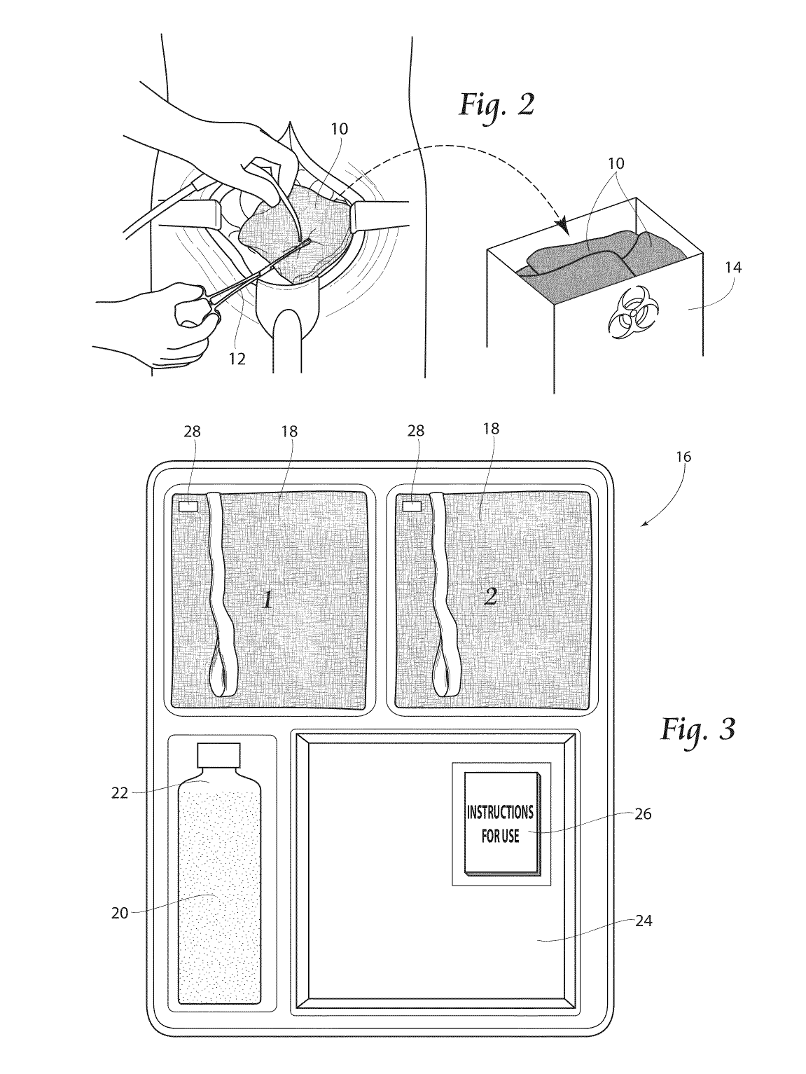

The method can further include (see FIG. 13) (xiii) at the end of the surgical procedure, discarding the fewer and limited number of surgical sponge-pad devices 18 as medical waste. A comparison of FIG. 2 (prior art) to FIG. 11 graphically demonstrates the significant reduction in environmental damage due to a reduction of surgical sponges comprising medical waste, as well as the contribution to lowered health care costs, that the technical features of the systems and methods provide. The systems and methods also lessen the problem of reconciling the surgical sponge count before wound closure, because there are only a limited number of surgical sponge-pad devices 18 to be accounted for and markedly reducing the time for sponge and needle counts following a procedure.

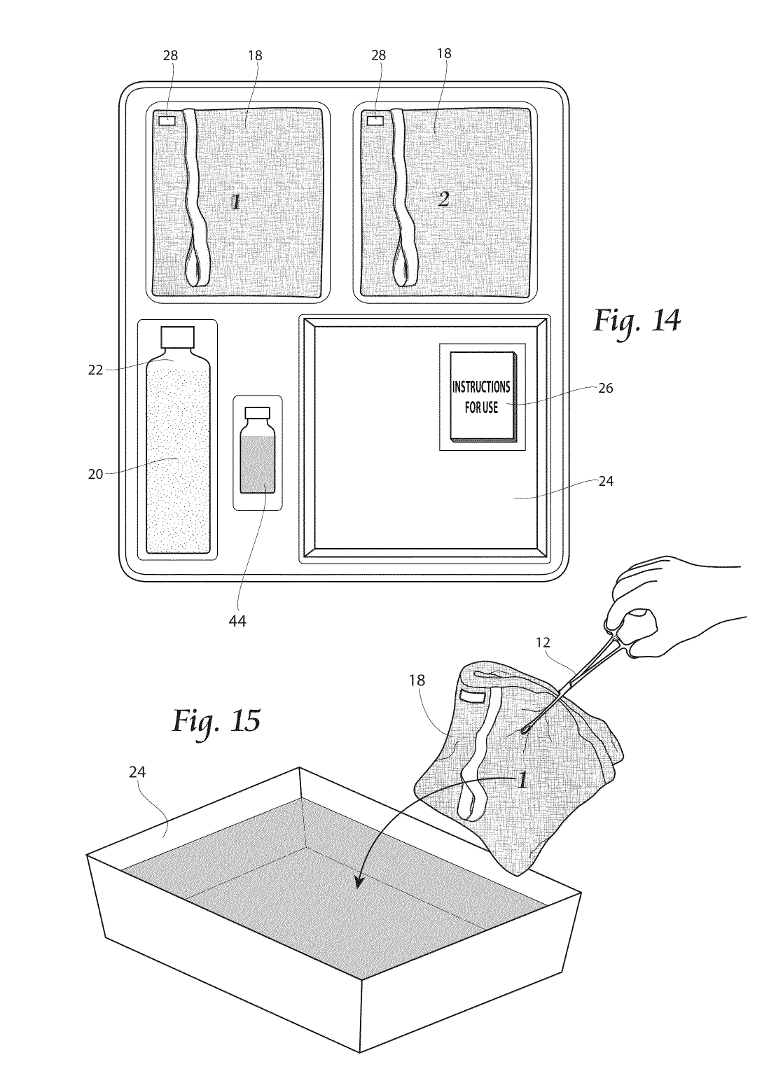

FIG. 14 shows the surgical sponge-pad system 16 for use during a single surgical procedure, with the system 16 further including a dye component 44. The dye component 44 allows the user to dye the sponge device 18 to a particular color desired by the user, which may be beneficial in performing particular procedures. The dye component 44 will be a non-toxic, biocompatible coloring that can be used internally and externally of a patient.

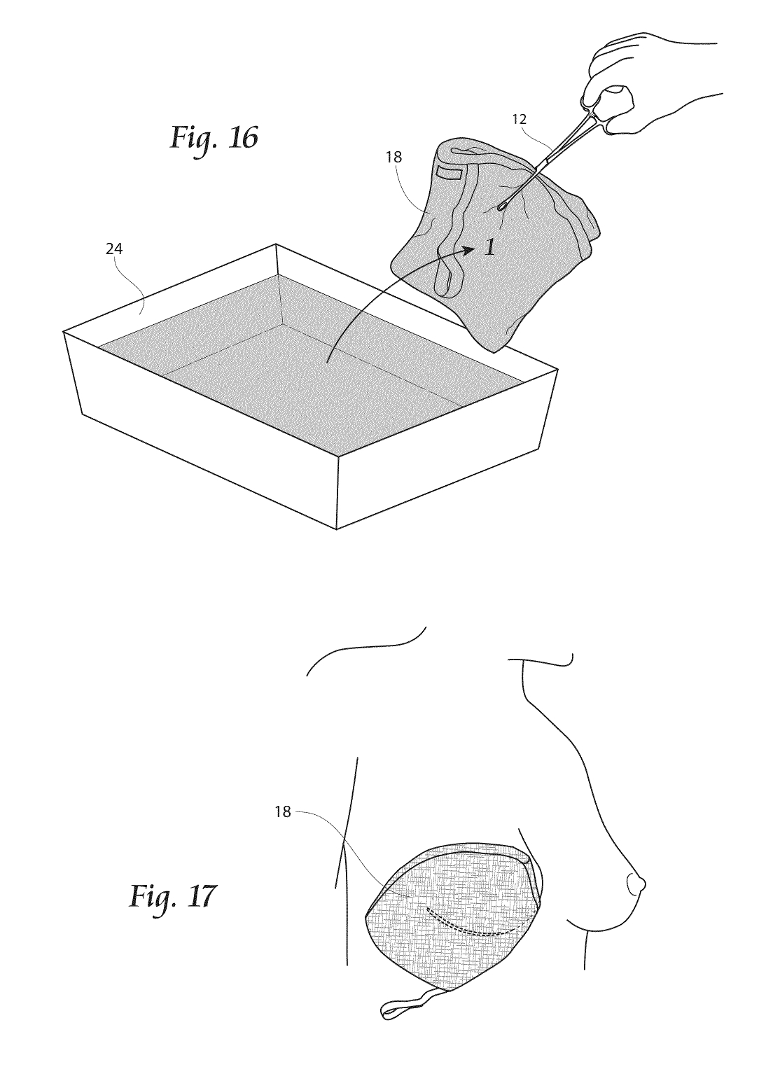

As FIGS. 15 and 16 show, the dye component 44 may be added to the conditioning liquid 20 within the tray 24. The sponge device 18 is then immersed in the tray 24 (FIG. 15) and removed (FIG. 16), thereby resulting in a colored surgical sponge device 18. The surgical sponge-pad device 18 will then be used as discussed above. It should be noted that the sponge-pad device 18 could be dyed a particular color prior to inclusion within the system 16 so that the sponge-pad devices 18 could be personalized for a particular hospital or doctor, and include a particular logo or pattern, if desired.

The surgical sponge-pad device 18 of the present invention can be utilized in a wide range of surgical procedures. For example FIG. 17 demonstrates the surgical sponge-pad device 18 being used during a procedure such as a mastectomy. The surgical sponge-pad device 18 can be used as a cover to keep the operating area moist and conditioned, as desired. That is, because the surgical sponge-pad device 18 of the present invention can retain a much more substantial amount of liquid and can also have the liquid squeezed out of the surgical sponge-pad device 18, the surgical sponge device 18 can be regulated to a particular general moisture content. The surgical sponge-pad also is nearly as absorbent when dry as when wrung out and moist. The sponge-pad device 18 could also be used as a tie-over bolster for skin grafts and to support and protect tissues following reconstructive surgery, e.g. nipple reconstruction surgery.

FIGS. 18-20 further demonstrate the utility and adaptability of the surgical sponge-pad device 18. An invasive surgical procedure that uses a sternotomy retractor 46 for spreading apart the chest is depicted. As shown in FIG. 18, the surgical sponge-pad device 18 is used internally of the body cavity, to absorb bodily fluids so that the surgical procedure may be carried out more efficiently. Such fluids may include blood, cerebrospinal fluid, bile, stomach acid, fecal material, or any other bodily fluid that should be minimized during a procedure. Furthermore, because of the greatly increased absorbency of the surgical-sponge-pad device 18 of the present invention, fewer surgical sponge-pad devices 18 need to be used, and they do not need to be rotated in and out of the body cavity as often, thereby providing a more efficient surgical process.

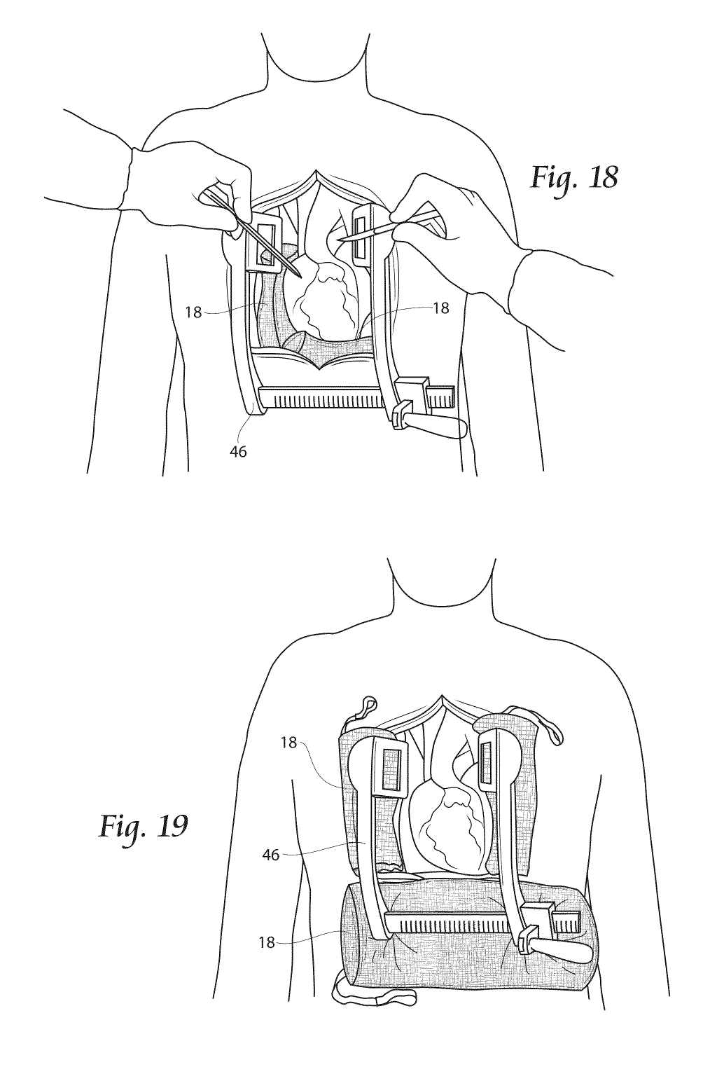

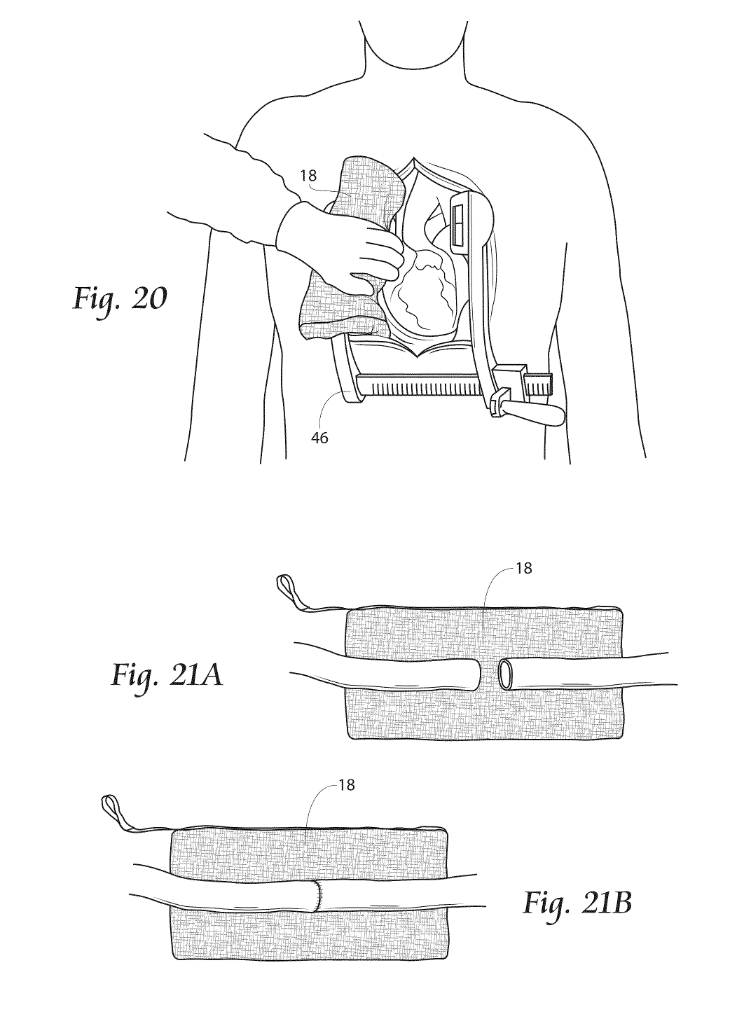

In FIG. 19, the surgical sponge-pad device 18 is used as insulation for the body against the retractor 46. The surgical sponge-pad device 18 is more durable than prior sponges and pads, which allows for the surgical sponge-pad device 18 to minimize any bruising or pain that may be associated with having the retractor 46 placed upon the patient. The durability of the surgical sponge-pad device 18 is also evidenced in FIG. 20, wherein the surgical sponge-pad device 18 is used to retract an internal organ, thereby allowing the surgeon better access to an operating area. The design and shape of surgical sponge-pad device 18 allows such retraction without fear of contaminating or damaging the retracted organ.

As FIGS. 21A and 21B, the surgical sponge-pad device 18 can be used as a backing surface when performing a vascular or microvascular anastomosis procedure. In such a situation, the surgical sponge-pad device 18 will allow for the vein, artery, or other body conduit to retain sufficient moisture and not to become dried out during the procedure, and in addition Lidocaine, Marcaine or other vasodialator may be used.

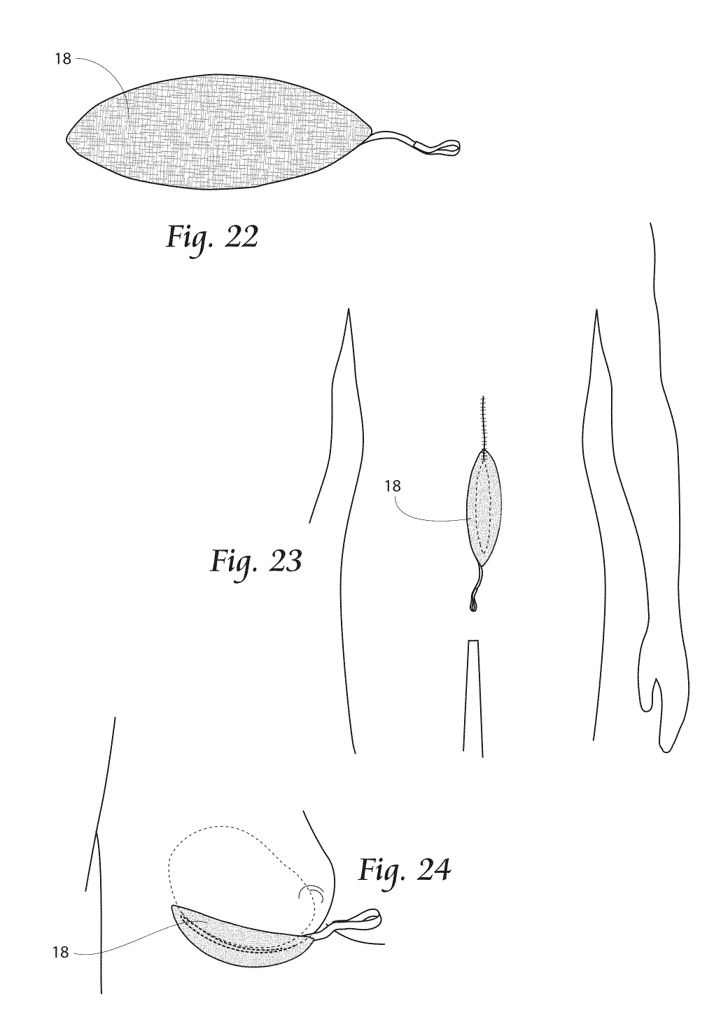

The surgical sponge-pad device 18 of the present invention can be of varying sizes and shapes, as is evident in FIGS. 22-24. The surgical sponge-pad device 18 is shown as a curved, oval shape, which is shaped to allow for easy withdrawal and may be temporarily placed inside to protect bowel or an implant during wound closure. Because of the durability of the surgical sponge-pad device 18, the pointed, oval shape could either be manufactured in such a shape, or could be cut to a particular shape while carrying out at the procedure itself, which would be particularly advantageous in certain first aid, trauma, and battlefield situations when a wound may need to be packed with the surgical sponge-pad device 18, and may also have some applications in space travel.

As shown in FIGS. 23 and 24, the oval surgical-sponge-pad device 18 particularly covers where stitching may occur, e.g. an abdominal incision (FIG. 23) or breast incision (FIG. 24). The surgical sponge-pad device 18 will cover the incision to prevent the tissue around the incision edges from drying out. Similarly, the surgical sponge-pad device 18 can also be used in post-operative situations to keep the wound area properly conditioned.

The multifunctional surgical sponge-pad will have significant and brand new applications in surgery for the protection and hydration of tissues, transmission of antibiotics and antimicrobials and other solutions, barriers in surgery and even use in transfer of transplanted organs as well as many other future medical and surgical uses yet to be determined. For example, the sponge-pad devices can be used in emergency room (ER) applications for open wounds to absorb blood and other bodily fluids, as well as keeping an area covered, clean, and moist when a trauma patient comes into the ER and must wait before being seen. Such similar uses could be used on the battlefield and in first aid trauma situations, wherein the sponge-pad device may keep the area clean and moist, as well as possibly delivering medicines to the patient, e.g. epinephrine, coagulants, antimicrobials, and antibiotics.

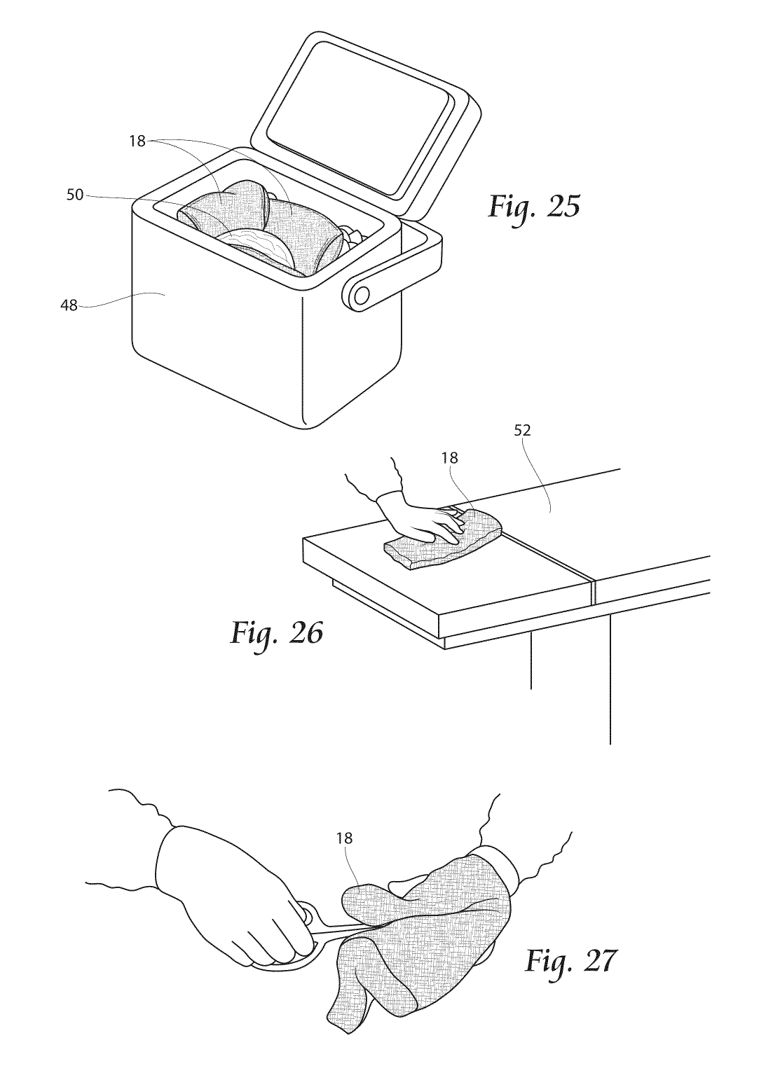

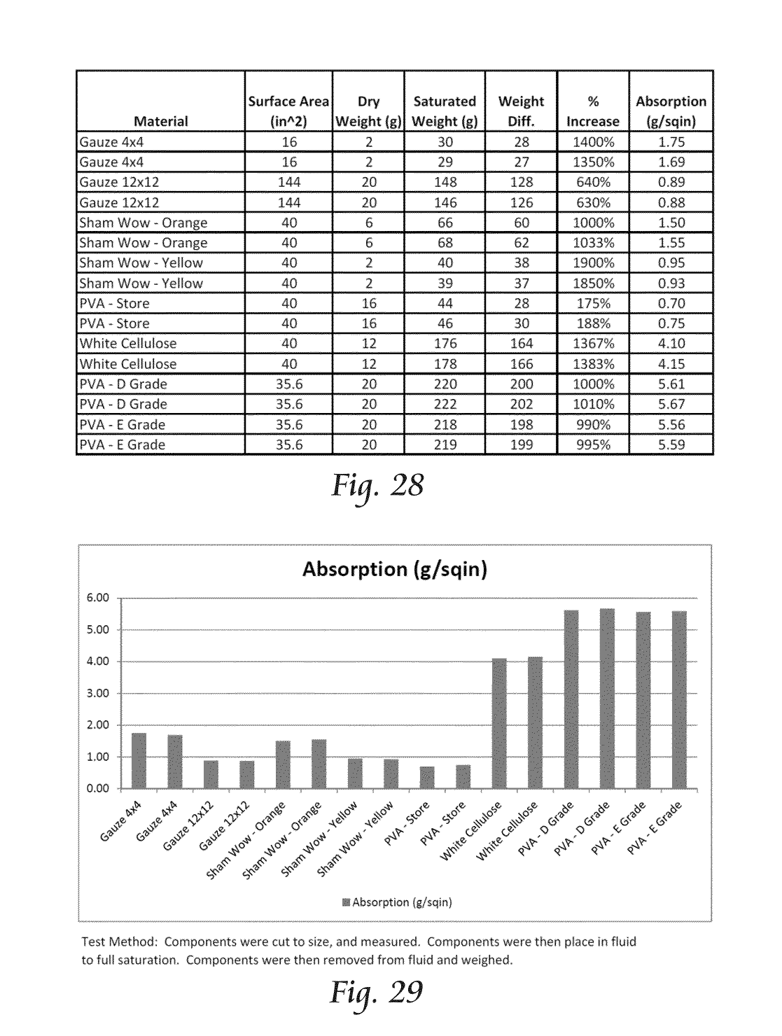

The adaptability and use of the present invention is further demonstrated in FIG. 25. A cooler 48 that is used to transport an organ 50 is shown such as a solid organ transplant operation. The surgical sponge-pad devices 18 allow for the organ 50 to be properly hydrated during the transportation of the organ.

As previously noted, the surgical sponge-pad device 18 could further incorporate antimicrobial or antifungal features. The surgical sponge-pad device 18 could also incorporate cleaners, soaps, waxes, etc., which would be beneficial when using the surgical sponge device 18 in general cleaning, as demonstrated in FIG. 26. Such use is also evident of the potential use of the surgical sponge-pad device 18 in industries and fields that require cleaning and sterilization, e.g. the food industry, cleaning industry, hospitals, athletic equipment use, and the like.

Likewise, the sponge-pad device 18 could be used for sterilization of surgical equipment, as shown in FIG. 27. Because of the durability of the sponge-pad devices 18, the sterilization could be done before and after a procedure, as well as using the sponge-pad device 18 during the procedure. Because the sponge-pad device 18 can be resterilized and reused during the procedure, biowaste is greatly reduced compared to prior art sponges. Further, the multiple uses for the present surgical sponge-pad device 18 significantly provides an advantageous device over prior art sponges, as it can be utilized in varying ways while retaining its integrity for each of the particular uses.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A reusable multi-use surgical sponge-pad for use during medical procedures comprising:
an absorbent material possessing a capability to quickly absorb bodily fluids, said absorbent material also possessing the capability of being rinsed and reused repetitively during the same procedure.

2. The surgical sponge-pad of claim 1, wherein the absorbent material is lint-free.

3. The surgical sponge-pad of claim 1, wherein the absorbent material is selected from the group consisting of: polyvinyl alcohol, polyurethane, and polyvinyl acetate.

4. The surgical sponge-pad of claim 3, wherein the absorbent material is polyvinyl acetate.

5. The surgical sponge-pad of claim 1 further comprising antibacterial material.

6. The surgical sponge pad of claim 1 further comprising an antimicrobial material.

7. The surgical sponge pad of claim 1, wherein the surgical sponge pad is capable of retaining fluid during the medical procedure, whereby the surgical pad sufficiently moistens exposed tissue during the medical procedure and keeps the exposed tissue from excessively drying out during the surgical procedure.

8. The surgical sponge pad of claim 7, wherein the surgical sponge pad is used for the transportation of an organ.

9. The surgical sponge pad of claim 1,
further capable of protecting and softening tissue margins and edges of open skin during the medical procedure.

10. The surgical sponge pad of claim 9,
wherein the surgical sponge pad protects and softens the tissue margins and edges of open skin that are in contact with a retractor used during the surgical procedure.

11. The surgical sponge pad of claim 1, where in the medical procedure is a microvascular or vascular procedure, whereby the surgical sponge pad is capable of receiving or containing vasodialator substances.

12. The surgical sponge pad of claim 1 wherein the surgical sponge pad is capable of walling off an internal organ during the surgical procedure.

13. The surgical sponge pad of claim 1 being capable of sequestering spilled bowel contents during the medical procedure, thereby minimizing infection and peritonitis during the medical procedure.

14. The surgical sponge pad of claim 1, wherein said surgical sponge pad is capable of wicking cerebrospinal fluid during neurosurgery or spinal surgery.

15. The surgical sponge pad of claim 1, further comprising an RFID tag.

16. The surgical sponge pad is sterilized and ready for intraoperative surgical use.

17. The surgical sponge pad may contain a standard sponge barium tag for X-ray examination incorporated into the pad.

18. A system for performing a medical procedure comprising:
a limited number of surgical sponge pads according to claim 1.

19. The system according to claim 15, further comprising:
an aqueous conditioning liquid; and
a conditioning tray for carrying said aqueous conditioning liquid, wherein said conditioning tray capable of receiving said sponges for submersion during said medical procedure.

20. A method for carrying out a medical procedure comprising the steps of:
(i) providing the surgical sponge-pad system of claim 16;
(ii) initially conditioning the surgical sponge pad of the surgical sponge-pad system in the conditioning liquid to initially condition said surgical sponge-pads for use;
(iii) removing the surgical sponge-pad device from the conditioning liquid and releasing residual liquid by manual wringing;
(iv) manipulating the conditioned surgical sponge-pad device in the operating field to absorb blood, body fluids, as well as water and other aqueous liquids in the operative site;
(v) returning the now-used surgical sponge-pad device to the conditioning liquid to clean and rinse it; and
(vi) repeating steps (i)-(v) as necessary.

21. The method of claim 20 further including the steps of:
(vii) providing a second surgical sponge-pad device in the surgical sponge-pad system;
(viii) conditioning said second surgical sponge-pad device for use by removing it from the conditioning liquid and removing residual liquid by manual wringing;
(ix) manipulating the second conditioned surgical sponge-pad device in the operating field to absorb blood, body fluids, as well as water and other aqueous liquids in the operative site;
(x) returning the now-used second surgical sponge-pad device to the conditioning liquid to clean and rinse it; and
(xi) repeating steps (vii)-(x) as necessary.

22. The method of claim 21 including the step of alternately using the first surgical sponge-pad device and the second surgical sponge-pad device.

23. The method of claim 20 further including the step of drying the surgical sponge-pad device.

24. The method of claim 20 further comprising the step of using said surgical sponge pad to clean a surgical instrument postoperative.

25. The method of claim 20 wherein the step of manipulating the surgical sponge pad further comprises walling of an internal organ during said medical procedure.

26. The method of claim 21 further comprising the steps of:
providing a third surgical sponge pad; and
protecting skin and soft tissue margins during said surgical procedure by placing said third surgical sponge pad between a surgical device used during said surgical procedure and said skin and soft tissue margins.

27. The method of claim 21 wherein the step of manipulating the surgical sponge pad further comprises using said surgical sponge pad to wick cerebrospinal fluid.

28. The method of claim 21 wherein the step of manipulating the surgical sponge pad further comprises using said surgical sponge pad to retain fluids sufficiently to moisten exposed tissue during the medical procedure, thereby preventing the exposed tissue from drying out.
